(19) United States
(12) Patent Application Publication
(10) Pub. No.: US 2008/0044452 A1
(43) Pub. Date: Feb. 21, 2008

(54) DRUG DELIVERY SYSTEM FOR TREATMENT OF TRANSITIONAL CELL CARCINOMA

(76) Inventor: Robert I. Carey, Sarasota, FL (US)

Correspondence Address: CARDINAL LAW GROUP, Suite 2000, 1603 Orrington Avenue, Evanston, IL, 60201, US

(21) Appl. No.: 11/829,680

(22) Filed: Jul. 27, 2007

Related U.S. Application Data
(60) Provisional application No. 60/833,737, filed on Jul. 27, 2006.

(51) Int. Cl. A61K 9/00; A61K 31/407; A61K 38/02; A61P 35/00; A61K 39/07

(52) U.S. Cl. 424/423; 424/246.1; 514/2; 514/410

(57) ABSTRACT

A system for treating tissue carcinomas and benign inflammatory conditions of the urinary tract includes a flowable medium containing a bioactive agent and a thermogelling aqueous polymer solution. The thermogelling aqueous polymer solution maintains the bioactive agent within the urinary tract for a therapeutically optimal period of time. One embodiment includes delivering a bioactive agent in a flowable medium through an orifice into a body cavity, contacting the tissue to be treated with the bioactive agent, and filling the orifice of the body cavity with a thermogelling aqueous polymer solution. The polymer solution maintains the bioactive agent in contact with the tissue to be treated for a therapeutically optimal period of time.

### RELATED APPLICATIONS

This application claims priority to and the benefit of U.S. Provisional Patent No. 60/833,737 filed on Jul. 27, 2006 titled Drug Delivery System for Treatment of Transitional Cell Carcinoma, the entirety of which is incorporated by reference.

### TECHNICAL FIELD

This invention relates generally to biomedical devices that are used for treating tissue carcinomas and benign inflammatory conditions. More specifically, the invention relates to a two part delivery system for site specific delivery of a bio-active agent that includes a flowable liquid medium and a thermosetting gel.

### BACKGROUND OF THE INVENTION

The urinary tract (FIG. 1) originates with kidneys 102, and includes ureters 104, urinary bladder 106 and urethra 108. As shown in FIG. 2, kidneys 102 have an outer portion, renal cortex 202, that surrounds and protects the inner structures of kidneys 102. Kidneys 102 receive blood through renal artery 206. In medulla 204 of kidney 102, the blood is filtered through the nephrons, a filtration system, that removes waste from the blood, regulates electrolyte balance, and forms urine. The blood then passes out of kidney 102 through renal vein 208. Once formed, urine flows through calyces 210, and collects in renal pelvis 212 and then flows out of kidneys 102 via ureters 104. The urine collects in urinary bladder 106, and finally, exits the body during urination via urethra 108. Urine is constantly formed, and depending on various physiological conditions, urine output ranges between 500 ml and 2 liters per day.

The interior surfaces of the tissues of the urinary tract are lined with a membranous layer of epithelial cells called the urethelium. A primary transitional cell carcinoma (TCC) is a carcinoma of the urethelium, and the terms “TCC” and “urothelial carcinoma” are used interchangeably in the urologic literature. Although TCCs occur most commonly in the urinary bladder, they can affect any part of the urinary collecting system (upper tract) including the renal pelvis, calyces, and ureters as well as the bladder and urethra.

Historically, nephroureterectomy has been considered the treatment of choice for TCCs of the upper tract if disease is not bilateral. However, with the development of percutaneous renal surgical techniques, ureteroscopy and laparoscopy, organ sparing and endoscopic treatment of upper tract TCCs have become common in medical practice. Such treatments include sharp dissection, laser, thermal, or cryo-ablation, and are often accompanied, especially in high risk patients, by adjuvant therapy consisting of intralumenal delivery of anticancer agents to prevent recurrence of the TCC.

Following removal of TCCs, aqueous solutions of anticancer agents are instilled directly in the urinary tract to destroy any remaining carcinoma cells and prevent recurrence of the disease. Anticancer agents appropriate for this use include: adriamycin, mitomycin, bleomycin, cisplatin, carboplatin, doxorubicin, daunorubicin, 5-fluouroacil, methotrexate, taxol, taxotere, and actinomycin D. However, because urine is constantly forming in the kidney, and flowing through the kidney pelvis and ureters, the drug solution is rapidly diluted and removed from the upper tract. Consequently, contact time between the anticancer agent and the carcinoma cells is frequently inadequate for the drug to be effective. Mitomycin C, the drug of choice for low grade urothelial carcinoma, is not stable in acidic urine, and the effectiveness of its delivery to the urothelium is known to be sensitive to pH, concentration, and dwell time. For high grade upper tract urothelial cancer, the preferred intraluminal treatment is an immunostimulatory agent Bacillus Calmette Guerin (BCG), an attenuated tuberculin organism typically delivered as an aqueous suspension.

Interstitial cystitis (IC) is a chronic inflammatory condition of the bladder wall. The etiology of IC is poorly understood and its symptoms of urinary urgency, frequency, and severe pelvic pain are often refractory to available treatment regimens, both systemic and intraluminal. Although the mortality due to IC is less than urothelial carcinoma, the morbidity for those affected is severe. Historically, those patients unable to achieve relief of their symptoms have been treated with cystectomy and urinary diversion. The most common intravesical treatment is dimethyl sulfoxide in aqueous solution; however, the severe urinary urgency, frequency and pain during treatment make it nearly impossible for the patients to retain the intravesical treatment in the urinary tract for an adequate dwell time without the aid of anesthesia.

It would be desirable, therefore, to provide a system for intraluminal delivery of bioactive agents that maintains the agent in contact with the tissue to be treated for an effective period of time, and would overcome the limitations and disadvantages inherent in the formulations described above.

### SUMMARY OF THE INVENTION

One aspect of the present invention provides a system for treating carcinomas of bodily organs comprising an effective amount of a bioactive agent in a flowable medium and a thermogelling aqueous polymer solution. When the bioactive agent is placed in a body cavity adjacent to the organ to be treated, the thermogelling aqueous polymer solution maintains the bioactive agent within the body cavity for a preselected length of time.

Another aspect of the present invention provides a method of treating a carcinoma of a tissue of a bodily organ comprising delivering a bioactive agent in a flowable medium through an orifice into a body cavity adjacent to the tissue to be treated, so that the bioactive agent contacts the tissue to be treated. The method further comprises filling the orifice of the body cavity with a thermogelling aqueous polymer solution and thereby maintaining the bioactive agent in contact with the tissue to be treated for a preselected period of time.

While the invention has been described with reference to particular embodiments, it will be understood by one skilled in the art that variations and modifications may be made in form and detail without departing from the spirit and scope of the invention.

### BRIEF DESCRIPTION OF THE DRAWINGS

FIG. 1 is a schematic illustration of the human urinary tract;

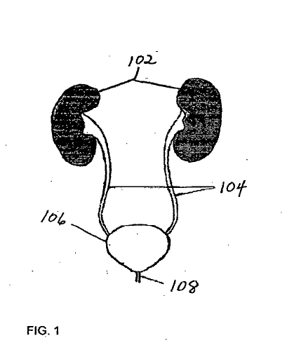

FIG. 2 is a schematic interior view of a human kidney showing the anatomic structures;

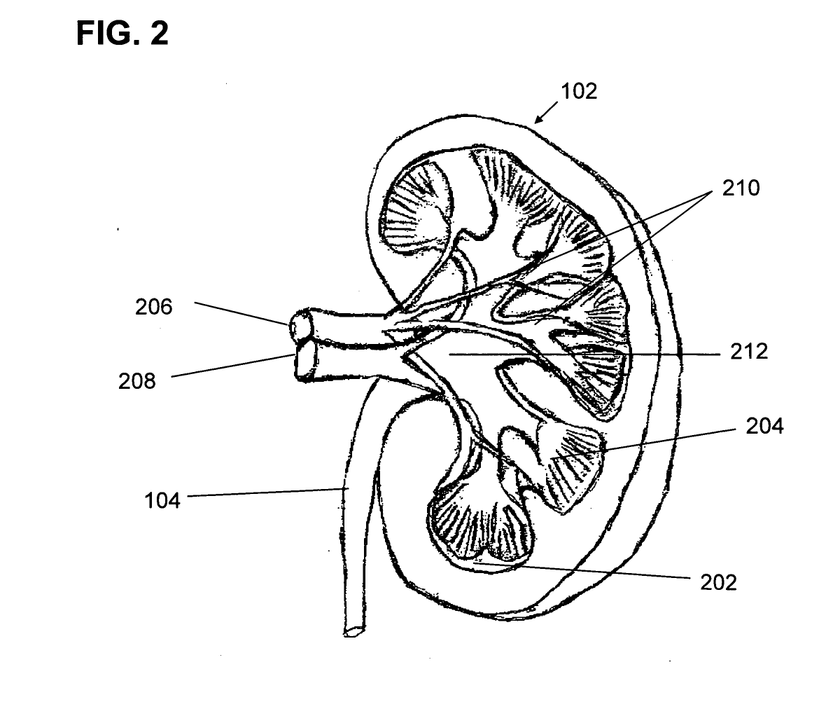

FIG. 3A is a schematic view illustrating the placement of a flowable medium including a bioactive agent and a thermogelling aqueous polymer solution in the urinary tract, in accordance with the invention;

FIG. 3B is a schematic representation illustrating the placement of a two phase delivery system for a bioactive agent, in accordance with the invention; and

FIG. 4 is a flow diagram of a method of treating a carcinoma in the urinary tract, in accordance with one aspect of the invention.

### DETAILED DESCRIPTION

Throughout this specification, like numbers refer to like structures.

The present invention comprises a system for delivering one or more bioactive agents to the urinary tract and maintaining the bioactive agent at a therapeutic concentration in contact with the tissue to be treated. A bioactive agent is meant to include any compound intended to alter a physiological condition or treat a disease. Examples of bioactive agents include diagnostic agents, drugs, prodrugs, proteins, peptides, and genetic material, including plasmids, nucleic acids and nucleic acid derivatives, DNA, RNA, recombinant DNA, recombinant RNA, and small interfering RNA (siRNA). The bioactive agent is either dissolved or suspended in an aqueous medium. Other substances such as ions, salts, buffering agents, solubilizing agents, suspending agents, solvents, or cosolvents may be added to the aqueous medium to maintain the solubility, stability, and availability of the bioactive agent in the aqueous medium.

In one embodiment, the viscosity of the aqueous medium is adjusted by adding at least one biocompatible, water soluble polymer to the aqueous medium. Examples of appropriate polymers include, but are not limited to, synthetic polymers such as polyethylene glycol, poloxamer, poloxamine, polyvinyl alcohol, polylactic acid, and naturally occurring polymers such as alginate, chitosan, and collagen. Adding these polymers, alone or in combination, to the aqueous medium increases the viscosity of the aqueous medium and, thereby reduces the rate at which the urine in the urinary tract mixes with the aqueous medium and dilutes both the aqueous medium and the bioactive agent contained within the medium. This in turn, extends the length of time that a therapeutic concentration of the bioactive agent is in contact with the tissue to be treated.

In some embodiments, polymers or polymer combinations are selected that form thermogelling solutions. The viscosities of such solutions change in a well defined manner as a function of temperature. Particularly useful for medical applications, such as drug delivery, are polymer solutions that are flowable liquids at ambient temperatures, ranging from about 10 C to about 30 C, but above a characteristic gelation temperature, the viscosity of the solution increases, so that the solution is no longer able to flow, and becomes a gel. Suitable polymers include polyoxyalkylene block copolymers such as poloxamers including poloxamer 407, poloxamer 338, poloxamer 288, poloxamer 238, or poloxamer 188, and poloxamines such as poloxamine 1107 and poloxamine 1307. For example, the viscosity of aqueous solutions of about 12% to about 25% (w/w) poloxamer 407 are flowable liquids at or below 20 C, but become highly viscous liquids or gels at or above about 30 C. By flowable liquid is meant a fluid substance having the ability to assume the shape of the space containing it. Such compositions may be applied to the target site by spraying, injection, or delivery through a catheter. Upon delivery, the solution flows over the target site and forms a coating on the target site and surrounding tissues. As the solution is warmed to body temperature, the viscosity of the solution increases, and when the gelation temperature is reached, the solution is no longer able to flow, and becomes a gel mass that adheres to the surface of the target site.

Because the polymers are water soluble, the gel mass slowly erodes as the polymer molecules on the surface of the mass dissolve into surrounding aqueous bodily fluids, such as blood, peritoneal fluid, extracellular fluid and urine. The rate at which the mass dissolves depends on the thickness and surface area of the gel mass, and the volume of fluid bathing the gel. In one embodiment, the fluid is urine, and the gel mass erodes within one to three hours.

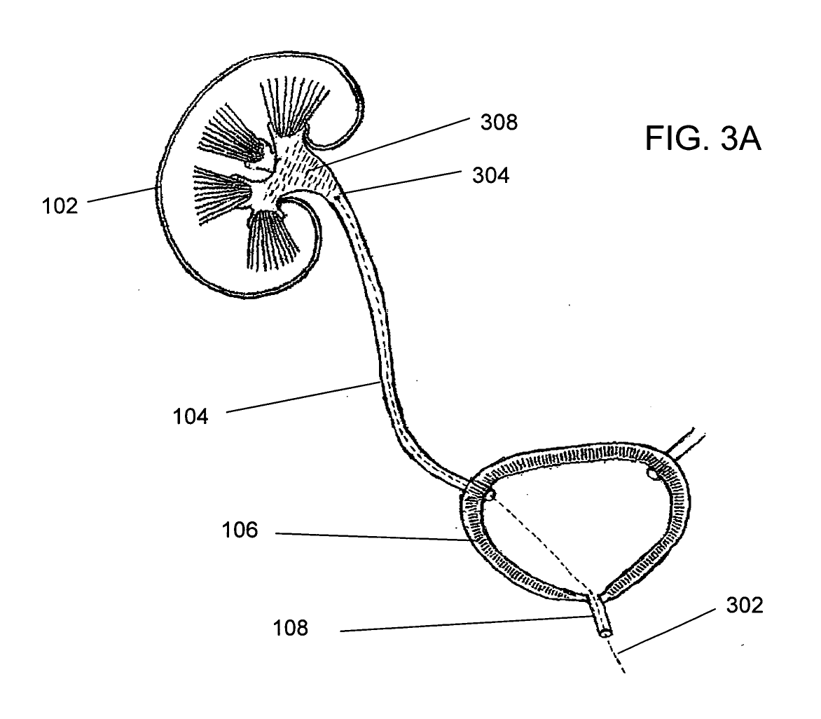

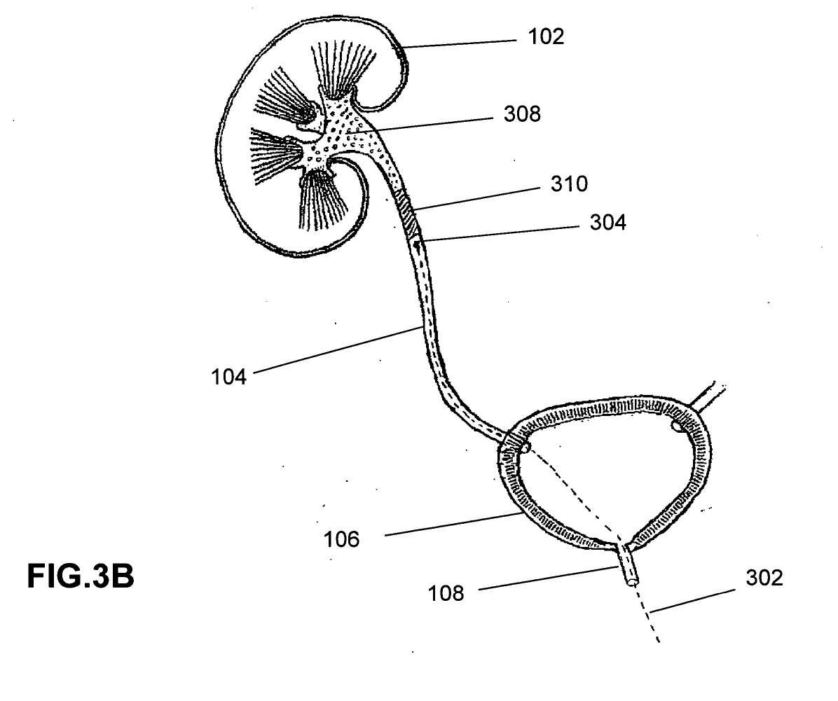

In one embodiment, a delivery medium is prepared by dissolving or suspending the bioactive agent in a flowable aqueous solution that is a nonflowable gel at body temperature. As shown in FIG. 3A, the solution is delivered to the target site within the urinary tract by catheter 302, a syringe or any other appropriate device. In this example, distal tip 304 of flexible catheter 302 is inserted through urethra 108, and advanced through bladder 106 and right ureter 104 and into renal pelvis 212 of right kidney 102. Upon delivery, the aqueous solution containing the bioactive agent forms gel mass 306 that coats the target site. In this embodiment, the target site is in the upper urinary tract and includes renal pelvis 212, calyces 210 and ureter 104. The bioactive agent contacts the target site for a defined period of time depending on the thickness of the gel coating. Finally, gel mass 306, containing the bioactive agent erodes from the outer surface, the gel dissolves into the urine and is removed from the body via the bladder and urethra by urine outflow.

Similarly, TCCs in urethra 108 may be treated by delivering the bioactive agent in a flowable liquid to the target site within urethra 108. As described above, the aqueous solution containing the bioactive agent forms a gel mass upon reaching body temperature, and remains where placed. After a defined treatment period, the gel is flushed from the urethra during urination.

In another embodiment, a two phase delivery system is used. The bioactive agent is dissolved or suspended in a liquid medium. As shown in FIG. 3B, using flexible catheter 302, liquid medium 308 is instilled in the upper urinary tract in contact with the tissue to be treated. Next, catheter 302 is partially withdrawn so that distal tip 304 is proximal to the liquid formulation, and a thermogelling composition is placed in the urinary tract, proximal to the liquid medium, where the thermogelling composition forms gel mass 310. Gel mass 310 remains in place for a defined period of time, between about one and about six hours, and prevents liquid medium 308 from being washed from the upper tract during that time. The length of time the gel mass remains in place is determined by the volume of the gel mass. During the drug delivery period, the gel mass is eroded by the urine bathing it, and is finally dissolved into the urine and flushed from the body. When gel mass 310 is no longer blocking ureter 104, liquid medium 308 is washed down ureter 104, and into bladder 106 where it is further diluted with urine, and finally is flushed from the body via urethra 108 during urination.

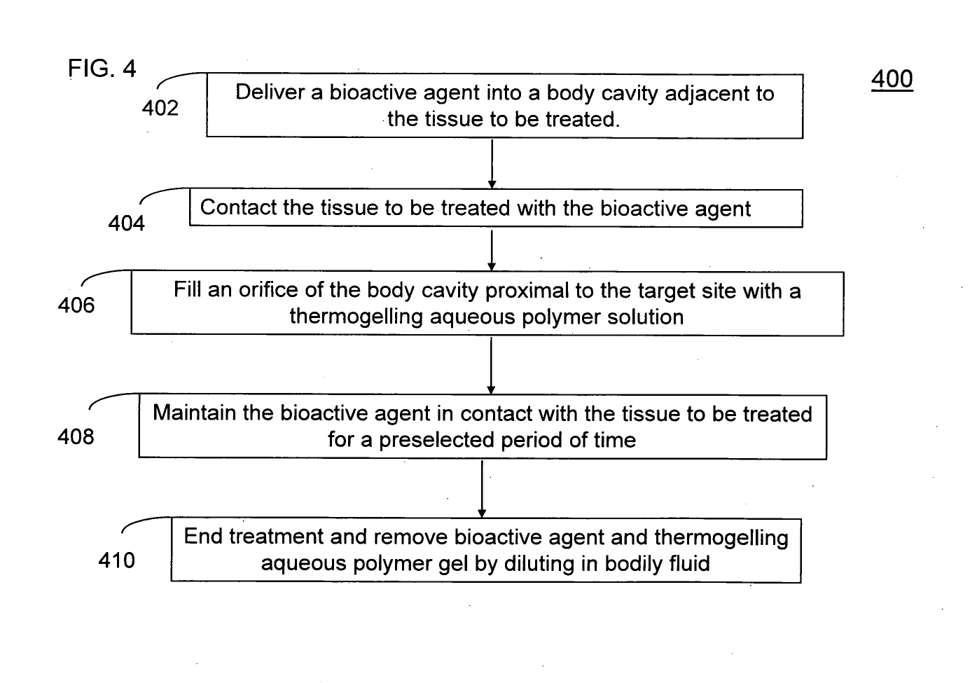

FIG. 4 is a flowchart illustrating method 400 for treating TCCs in the urinary tract, in accordance with the present invention. The distal portion of delivery catheter 302 is inserted into the urinary tract of a patient and is advanced so that distal tip 304 is adjacent to the target site in the upper urinary tract such as the renal pelvis or the distal ureter. A bioactive agent in a fluid medium is then delivered into the upper urinary tract from distal tip 304 of catheter 302, as indicated in Block 402. The fluid containing the bioactive agent bathes the target site and provides contact between the bioactive agent and the treatment site (Block 404).

Next, catheter 302 is withdrawn slightly so that distal tip 304 is proximal to the fluid medium containing the bioactive agent. A thermogelling aqueous polymer solution is then delivered into the urinary tract so that the gel fills the orifice of the body cavity proximal to the fluid medium, as indicated in Block 406. This orifice may be the ureter or the junction between the ureter and the kidney. The gel mass blocks the ureter and prevents the bioactive agent from flowing down the ureter, and maintains the bioactive agent in the renal pelvis or distal ureter, as indicated in Block 408. However, urine is constantly forming in the kidney, and bathing the surface of the gel mass. This in turn causes the gel to erode, and after a period of time depending on the size of the gel mass, the ureter is reopened. The bioactive agent is then washed down the ureter, through the bladder and out of the body, ending treatment, as indicated in Block 410.

### EXAMPLES

### Example I

Poloxamer 407 was dissolved in water to prepare a 20% w/w solution. Approximately 20 cc of the poloxamer solution was placed in a flexible plastic tube having a diameter similar to an adult human ureter and maintained at 37 C. The poloxamer solution quickly formed a gel mass. Normal saline solution was added to the system at a rate of 0.5 ml per minute to simulate the formation of urine. The surface of the gel mass was bathed in the increasing volume of saline, and the gel mass slowly eroded. After approximately 1.5 to 2 hours, the gel mass was entirely eroded, and the saline solution flowed freely down the tube.

Example II

Poloxamer 407 was dissolved in water to a 20% w/w ratio and mixed with methylene blue to form a miscible dark blue liquid. This liquid was injected using a 20 mL syringe through a 5 French Pollack catheter into a freshly harvested porcine ureter and kidney. The ureter and kidney were distended to fullness with a total volume of 15 mL and this was kept at 37 C in an aqueous medium. The mixture slowly seeped out of the ureteral orifice, during a period of approximately 180 minutes for complete evacuation. If a solution of 37 C normal saline is infused at a rate of 0.5 mL per minute via a 22 gauge needle placed into the renal collecting system, the mixture is completely evacuated within about 100 minutes.

### Example III

Poloxamer 407 was dissolved in water to a 20% w/w ratio and mixed with methylene blue to form a miscible dark blue liquid. This liquid was injected using a 20 mL syringe through a 5 French Pollack catheter into a freshly harvested porcine ureter and kidney. The ureter and kidney were distended to fullness with a total volume of 15 mL, and maintained at 37 degrees in an aqueous medium. A solution of 37 C normal saline was infused at a rate of 0.5 mL per minute via a 22 gauge needle placed into the renal collecting system. The mixture slowly seeped out of the ureteral orifice, requiring at least 180 minutes for complete evacuation.

### Example IV

Poloxamer 407 was dissolved in water to a 20% w/w ratio and mixed with methylene blue to form a miscible dark blue liquid. This liquid was injected using a 20 mL syringe through a 5 French Pollack catheter into a freshly harvested porcine ureter and kidney. The ureter and kidney were distended to fullness with a total volume of 15 mL and this was kept at 37 degrees in an aqueous medium for five minutes. At this point a 5 French Pollack catheter was passed through the gel into the renal pelvis and cold saline was infused via a 20 mL syringe. After three minutes the kidney and ureter were cut open through the long axis from lateral to medial exposing the cortex and renal collecting system. No residual gel was observed.

### Example V

Poloxamer 407 was dissolved in water to a 20% w/w ratio and mixed with methylene blue to form a miscible dark blue liquid. This liquid was injected using a 20 mL syringe through a 5 French Pollack catheter into a freshly harvested porcine ureter and kidney. The ureter and kidney were distended to fullness with a total volume of 15 mL and were kept at 37 degrees in an aqueous medium for five minutes. At this point, the kidney and ureter were cut open through the long axis from lateral to medial exposing the cortex and renal collecting system. Visual inspection confirmed that the gel was present throughout the entire collecting system. All parts of the urothelium within the kidney and ureter were in contact with the gel.

### Example VI

Poloxamer 407 was dissolved at 25% weight/weight formulation and injected into the lower portion of a vertical 6 mm inner diameter clear tube to form a 3 cm clear plug. A solution of 0.25% methylene blue in normal saline was instilled at 37 C into the upper portion of the tube forming a column height of 20 cm, overlaying the gel layer. The plug and solution were kept at 37 C and each remained in place for 135 minutes after which time the plug dissolved into the solution, and the solution flowed out of the tube.

### Example VII

Poloxamer 407 was dissolved in water at 25% weight/weight formulation and injected into the lower portion of a 6 mm inner diameter clear tube to form a 3 cm clear plug. A solution of 0.25% methylene blue in normal saline was instilled at 37 C into the upper end of the tube for a column height of 20 cm over laying the plug. The plug and solution were maintained at 37 degrees C., while normal saline (37 C) was continuously infused at the top of the column at a rate of 0.5 mL per minute. The plug and column remained in place for periods of time that varied between 70 minutes and 110 minutes, depending on the rate at which small amounts of fluid flowed over the plug and the rate at which the column height increased. At no point did the plug remain in place if the column height reached 35 cm or greater.

While the invention has been described with reference to particular embodiments and examples, it will be understood by one skilled in the art that variations and modifications may be made in form and detail without departing from the spirit and scope of the invention.

1. A system for treating a carcinoma of a bodily organ comprising:
an effective amount of a bioactive agent in a flowable medium; and
a thermogelling aqueous polymer solution wherein when the bioactive agent is placed in a body cavity, the thermogelling aqueous polymer solution maintains the bioactive agent within the body cavity for a preselected period of time.

2. The system of claim 1 wherein the thermogelling aqueous polymer solution comprises a polyoxyalkylene block copolymer in an aqueous solution.

3. The system of claim 2 wherein the polyoxyalkylene block copolymer is a poloxamer.

4. The system of claim 3 wherein the polyoxyalkylene block copolymer is poloxamer 407, poloxamer 338, poloxamer 288, poloxamer 238, or poloxamer 188.

5. The system of claim 2 wherein the polyoxyalkylene block copolymer is a poloxamine.

6. The system of claim 5 wherein the polyoxyalkylene block copolymer is poloxamine 1107 or poloxamine 1307.

7. The system of claim 2 wherein the concentration of the polyoxyalkylene block copolymer is selected to provide a solution that forms a gel below about 35 C.

8. The system of claim 1 wherein the organ to be treated is selected from a group consisting of kidney, ureter, urinary bladder and urethra.

9. The system of claim 1 wherein the carcinoma to be treated is a transitional cell carcinoma.

10. The system of claim 1 wherein the bio-active agent forms a solution or a suspension in the flowable medium.

11. The system of claim 10 wherein the flowable medium comprises water and a polyoxyalkylene block copolymer.

12. The system of claim 11 wherein the flowable medium further comprises solvents, cosolvents, solubilizing agents, suspending agents, and buffering agents selected to optimize the bioavailability of the bioactive agent.

13. The system of claim 10 wherein a buffer maintains the pH of the flowable medium and the thermogelling aqueous polymer solution is adjusted to optimize the bioactivity of the bioactive agent.

14. The system of claim 1 wherein the bioactive agent is an anti-cancer agent selected from the group consisting of Mitomycin C, Bacillus Calmette Guerin, adriamycin, mitomycin, bleomycin, cisplatin, carboplatin, doxorubicin, daunorubicin, 5-fluouroacil, methotrexate, taxol, taxotere, and actinomycin D.

15. The system of claim 1 wherein the bioactive agent is selected from the group consisting of peptides, proteins, nucleic acid derivatives, and small interfering RNA (siRNA).

16. The system of claim 1 wherein the preselected time is between about 30 minutes and about 6 hours.

17. A method of treating a carcinoma of a tissue of a bodily organ comprising:
delivering a bioactive agent in a flowable medium through an orifice into a body cavity adjacent the tissue to be treated;
contacting the tissue to be treated with the bioactive agent;
filling the orifice of the body cavity with a thermogelling aqueous polymer solution; and
maintaining the bioactive agent in contact with the tissue to be treated for a preselected period of time.

18. The method of claim 17 further comprising diluting the thermogelling aqueous polymer solution in a bodily fluid and thereby removing the thermogelling aqueous polymer solution from the bodily orifice.

19. The method of claim 17 further comprising determining the length of time the bioactive agent contacts the tissue to be treated by the amount of thermogelling aqueous polymer solution placed in the orifice of the body cavity.

20. The method of claim 17 wherein the body cavity is an interior lumen of an organ selected from a group consisting of kidney, ureter, urinary bladder and urethra.

21. The method of claim 17 wherein the carcinoma to be treated is a transitional cell carcinoma.

22. The method of claim 17 wherein the bioactive agent is an anti-cancer agent selected from the group consisting of Mitomycin C, Bacillus Calmette Guerin, adriamycin, mitomycin, bleomycin, cisplatin, carboplatin, doxorubicin, daunorubicin, 5-fluouroacil, methotrexate, taxol, taxotere, and actinomycin D.

23. The method of claim 17 wherein the bioactive agent is selected from the group consisting of peptides, proteins, nucleic acid derivatives, and small interfering RNA (siRNA).
